(19) United States
(12) Patent Application Publication
(10) Pub. No.: US 2026/0153491 A1
(43) Pub. Date: Jun. 4, 2026

(54) MONITORING CANNABIS PLANTS

(71) Applicant: SIGNIFY HOLDING B.V., EINDHOVEN (NL)

(72) Inventors: MARCELLINUS PETRUS CAROLUS MICHAEL KRIJN, EINDHOVEN (NL); CÉLINE CATHERINE SARAH NICOLE, EINDHOVEN (NL); LEONIE MARIA GEERDINCK, EINDHOVEN (NL)

(21) Appl. No.: 18/707,672

(22) PCT Filed: Nov. 3, 2022
(86) PCT No.: PCT/EP2022/080706

§ 371 Date: May 6, 2024

(30) Foreign Application Priority Data
Nov. 9, 2021 (EP) 21207052.8

(51) Int. Cl. G01N 33/00

(52) U.S. Cl. CPC G01N 33/0098; G01N 33/0047

(57) ABSTRACT

A mechanism for monitoring cannabis and predicting or determining a harvesting time. A sensing arrangement generates a temporal sequence of VOC values, each representing a measured amount of a first set of one or more volatile organic compounds (VOCs) in the air surrounding some cannabis. This temporal sequence is then processed to determine a harvesting time, at which the cannabis is ready for harvesting (e.g., meets some predetermined criteria for achieving a harvesting goal).

### FIELD OF THE INVENTION

The present invention relates to the field of plant monitoring, and in particular, to the monitoring of Cannabis.

### BACKGROUND OF THE INVENTION

Plants of the genus Cannabis (“cannabis”) are flowering annual plants, which includes at least the species Cannabis sativa. Cannabis indica and Cannabis ruderalis are either seen as sub-species/varieties of Cannabis sativa, or as separate species (under the genus Cannabis) in their own right.

Cannabis flowers produce valuable phytochemicals as a by-product, such as terpenes and cannabinoids (such as THC and CBD). It is known to use several of these phytochemicals to relieve the symptoms of a number of medical conditions, such as relieving pain and/or preventing nausea. There is a worldwide interest in the medical use of cannabis, and an increasing trend towards legalizing the medical use of cannabis.

It is usually considered necessary to grow cannabis under controlled circumstances to be able to guarantee a sufficient quality for medicinal purposes. For this reason, growth mostly takes place in greenhouses (i.e., with daylight contributions) or indoor (i.e., without daylight). For improved and repeatable quality, e.g., improved and reproducible phytochemical content, supplemental light (e.g., provided by an LED arrangement) is often used.

A typical growth cycle of a cannabis plant in a commercial greenhouse or indoor setting consists of several distinguishable growth phases. Plants begin in the seedling phase, in which young plants are propagated from seeds or from cuttings taken from a female mother plant. Plants then move to the vegetative phase, in which the female plants are transplanted to a lower plant density and grown to a certain degree of maturity. This is called the vegetative phase. After the vegetative phase, there is a flowering phase that starts with a transition to the reproductive phase (i.e., flowering). Cannabis plants are so-called short-day plants. They start flowering when the photoperiod is shortened. To induce flowering, the photoperiod is shortened to typically 12 hours per day. At the end of the flowering phase, the flowers are harvested (a destructive process).

One way to improve the quality of the harvested flowers is to correctly time the harvesting of the flowers (as the amount of phytochemical content depends upon a time of harvest).

Determining an appropriate time of harvest is currently performed by visual inspection of the flowers (e.g., using a magnifying glass). The appearance of the trichomes, the small resin glands on flowers, is one way to identify a good time for harvesting. Another indicator is the pistils turning orange-brown at maturity. An appropriate time for harvesting is when about half of the pistils are shaded orange-brown.

However, these approaches rely upon an intensive and experience-sensitive approach for identifying an appropriate time for harvest. There is therefore a desire for an accurate and repeatable mechanism for monitoring Cannabis plants to identify an appropriate time for harvesting.

In addition, as harvesting of cannabis plants, especially the cannabis flowers, and the post-harvest processing thereof is a labor-intensive activity, growers are looking for solutions enabling an improved (e.g., better-timed) predicting of the time of harvest allowing an improved (e.g., better-timed) scheduling of the accompanying labor for this harvest.

WO 2019/237200 A1 relates to a precision agriculture system and related methods. There is disclosed a growing system comprising: a plurality of sensors for sensing one or both of parameters of a plant or parameters of an environment in which the plant is being grown; an environmental control system for controlling one or more growing conditions of the plant; a controller coupled to the plurality of sensors and configured to: receive sensor data from the plurality of sensors; determine whether parameters based at least in part on the sensor data match one or more performance criteria; and cause the environmental control system to perform an adjustment to at least one growing condition of the plant in response to a determination that the parameters do not match the one or more performance criteria.

### SUMMARY OF THE INVENTION

The invention is defined by the claims.

According to examples in accordance with an aspect of the invention, there is provided a method for monitoring cannabis plants.

The method comprises: monitoring, using a sensor arrangement and over a period of time, an amount of a first set of one or more volatile organic compounds, VOCs, in air within the vicinity of the cannabis plants to thereby generate a first temporal sequence of monitored VOC values, each VOC value representing a measure of the first set of one or more VOCs at a different point in time; and determining, based on the first temporal sequence of VOC values, a harvesting time for the monitored cannabis plants.

This disclosure proposes an approach for determining a harvesting time for monitored cannabis plants. An amount of VOCs in the air (e.g., the concentration of the first set of one or more VOCs) surrounding or in the vicinity of the cannabis plants (i.e., released by the cannabis) is iteratively measured, to produce a first temporal sequence of monitored VOC values. The first temporal sequence of VOC values is then processed to determine the harvesting time for the monitored cannabis plants.

Embodiments are based on the realization that a trend in the amount of VOCs in the air around a cannabis plant changes as the cannabis plant approaches maturity (i.e. becomes harvest-ready). In other words, the VOC signature of the air close to the cannabis plants represents a harvest-readiness of the VOC. More particularly, it is recognized that the sought-after phytochemical compounds of cannabis flowers, such as terpenes and cannabinoids (such as THC and CBD), are volatile and a measurable fraction of these are released into the air. This recognition is exploited in the present disclosure to identify when the cannabis plant is harvest ready.

It is appreciated that many VOCs released into the air differ from the volatiles of the sought-after compounds in the flowers, however, as long as these VOCs have a relation to the sought-after compounds (which is a reasonable assumption to make due to the growth characteristics of cannabis plants and/or cannabis flowers), they are candidates to be monitored and analyzed.

Embodiments thereby provide a reliable and automated mechanism for assessing when a cannabis plant is ready for harvest, e.g., when the cannabis flowers exhibit a suitable or optimum amount of desired phytochemical content.

The step of determining a harvesting time comprises determining a harvesting time based on one or more temporal patterns within the first temporal sequence of VOC values. This embodiment recognizes that patterns or trends within the first temporal sequence of VOC values indicate when a cannabis plant is approaching readiness for harvesting. By identifying these specific patterns or trends (e.g., increasing presence of VOCs or particular VOCs) then an accurate assessment of harvesting time can be generated.

The first set of one or more volatile organic compounds comprises one or more terpenes and/or cannabinoids. For instance, the first set of one or more volatile organic compounds may comprise only the one or more terpenes and/or cannabinoids.

The first set of one or more volatile organic compounds may comprise one or more monoterpenes and/or sesquiterpenes.

It has been identified that these VOCs are particularly responsive to a harvest-readiness of cannabis plants, as they correspond to the desired phytochemical content of cannabis plants, specifically cannabis flowers, for medicinal or treatment purposes.

The method may further comprise monitoring, using the sensor arrangement and over the period of time, an amount of a second set of one or more volatile organic compounds, VOCs, in air within the vicinity of the cannabis plant to thereby generate a second temporal sequence of monitored VOC values, each VOC value of the second temporal sequence temporally corresponding to a respective VOC value of the first temporal sequence and representing a measure of the second set of one or more VOCs; and processing the first and second temporal sequences to produce a third temporal sequence, each value of the third temporal sequence being temporally associated with a pair of respective values from the first temporal sequence and the second temporal sequence and being derived from the respective values of the first temporal sequence and the second.

In some preferred examples, the step of determining a harvesting time for the monitored cannabis plant comprises determining the harvesting time for the monitored cannabis plant based on the third temporal sequence. In particular, temporal patterns within the third temporal sequence can be used to identify when the cannabis plant is approaching harvest-readiness.

Optionally, each value of the third temporal sequence is equal to the ratio of the respective value of the first temporal sequence and the second temporal sequence. Using a ratio in this way provides a more robust and consistent mechanism for determining the harvesting time, as the ratio will be independent of proximity of the sensor arrangement to the plants and/or environmental conditions.

In some examples, the first set of one or more volatile organic compounds comprises all volatile organic compounds. Thus, a non-specific VOC sensor could be used, which can save resources and complexity of the system.

In some examples, the first temporal sequence of monitored VOC values comprises VOC values obtained within a predetermined temperature range only.

An amount of VOC released by a cannabis plant can be affected by air temperature. Generally, the higher the temperature, the more VOCs are released. By restricting the VOC values to be obtained within a predetermined temperature range only, then a more consistent or reliable analysis of the harvest-readiness of the cannabis plant can be determined.

For instance, the first temporal sequence of monitored VOC values may comprise VOC values obtained during daylight hours only or during nighttime hours only. Restricting the first temporal sequence to only contain values obtained during daylight or nighttime hours defines a range of temperatures between which the VOC values are obtained. This is because a temperature difference between different times of day alone or night alone is less than a temperature difference between the day and the night.

In some examples, each VOC value in the first temporal sequence of monitored VOC values is obtained after a respective manipulation, such as a respective insonification and/or vibration, of the cannabis plant.

An active manipulation of the cannabis plant releases additional VOCs into the air surrounding the cannabis plant. This increases a signal-to-noise ratio of any measured VOCs, to thereby improve the identification of when the cannabis plant is harvest-ready. Preferably, the manipulation is automated and therefore repeatable, to ensure that the cannabis plant undergoes a same manipulation for the purposes of increased consistency and reliability of the VOC measurements.

There is also proposed a method of controlling a growth environment for cannabis plant, the method comprising: obtaining a desired time for harvesting the cannabis plant; performing any previously described method to determine a harvesting time for the cannabis plant; and responsive to a difference between the determined harvesting time and the desired time, modifying one or more properties of the growth environment for the cannabis plant.

The one or more properties of the growth environment may comprise a temperature, a moisture level, a spectrum of light, a duration of light and/or a magnitude of light. These properties have been identified as influential on the growth/maturation speed of cannabis, such that modifying these properties in particular facilitates control over the growth conditions and the harvesting time of the cannabis.

There is also proposed a method comprising performing a plurality of instances of any previously described method, each instance being performed at a different location and/or height relative to the one or more cannabis plants.

There is also proposed a computer program product comprising computer program code means which, when executed on a computing device having a processing system, cause the processing system to perform all of the steps of any herein described method.

There is also proposed a cannabis monitoring system comprising a sensor arrangement configured to monitor, over a period of time, an amount of a first set of one or more volatile organic compounds, VOCs, in air within the vicinity of the cannabis plant to thereby generate a first temporal sequence of monitored VOC values, each VOC value in the sequence representing a measure of the first set of one or more VOCs at a different point in time; and a processing arrangement, communicatively coupled to the sensor arrangement, and configured to determine, based on the first temporal sequence of VOC values, a harvesting time for the monitored cannabis plant.

There is also proposed a system comprising the cannabis monitoring system and an environment modification system. The environment modification system is configured to modify a growth environment for the cannabis plant by obtaining a desired time for harvesting the cannabis plant; obtaining (from the cannabis monitoring system) a determined harvesting time for the monitored cannabis plant; and responsive to a difference between the determined harvesting time and the desired time, modifying one or more properties of the growth environment for the cannabis plant.

The environment modification system may comprise any suitable environment modifying device, such as a lighting arrangement, a temperature-controlling arrangement (e.g., air-conditioning or cooling systems and/or heating systems), a humidity controller (e.g., a humidifier and/or a dehumidifier) and so on.

In the context of the present disclosure, harvesting “cannabis” may mean harvesting a cannabis plant, harvesting cannabis flowers of the cannabis plant or harvesting a number of cannabis plants to which the VOC measurements are relevant.

In the context of the present disclosure, the term “determining” a harvesting time may also mean estimating or predicting a harvesting time.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates a system according to an embodiment;

FIG. 2 illustrates an example signature generated by a gas-chromatography sensor;

FIG. 3 illustrates a relationship between total amount of VOCs released by cannabis and time;

FIG. 4 illustrates two example signatures produced by a gas-chromatography sensor at different points during a cannabis plants growth or maturation;

FIG. 5 illustrates a method according to an embodiment; and

FIG. 6 illustrates another method according to an embodiment.

### DETAILED DESCRIPTION OF THE EMBODIMENTS

The invention will be described with reference to the figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the figures are merely schematic and are not drawn to scale. It should also be understood that the same reference numerals are used throughout the figures to indicate the same or similar parts.

The invention provides a mechanism for monitoring cannabis plants and predicting or determining a harvesting time. A sensing arrangement generates a temporal sequence of VOC values, each representing a measured amount of a first set of one or more volatile organic compounds (VOCs) in the air surrounding some cannabis plants. This temporal sequence is then processed to determine, predict or estimate a harvesting time, at which the cannabis plant is ready for harvesting (e.g., meets some predetermined criteria related to a harvesting goal).

Embodiments are based on the realization that the amount of certain VOCs, and the relationship between different VOCs released by cannabis plants in the air, changes during the course of their lifecycle. It is therefore possible to predict a harvesting time based on temporal trends or patterns in monitored VOC values. This provides useful information for identifying when to harvest the cannabis plants and/or for controlling an environmental control system for providing the environmental conditions for the cannabis plant to target a growth of the cannabis plant such that the cannabis plant is ready for harvest at a desired point in time.

Embodiments may be employed in cannabis growth systems, such as those used for growing medicinal cannabis and/or cannabis which is processed to extract desired ingredients for medication.

In the context of the present invention, the term “based on” indicates there is a direct causal relationship between a first and second element, such that a first element that is based on a second element is responsive to changes in the second element. More specifically, the term “based on” may be appropriately replaced by the term “by processing” (or grammatical variations of the same).

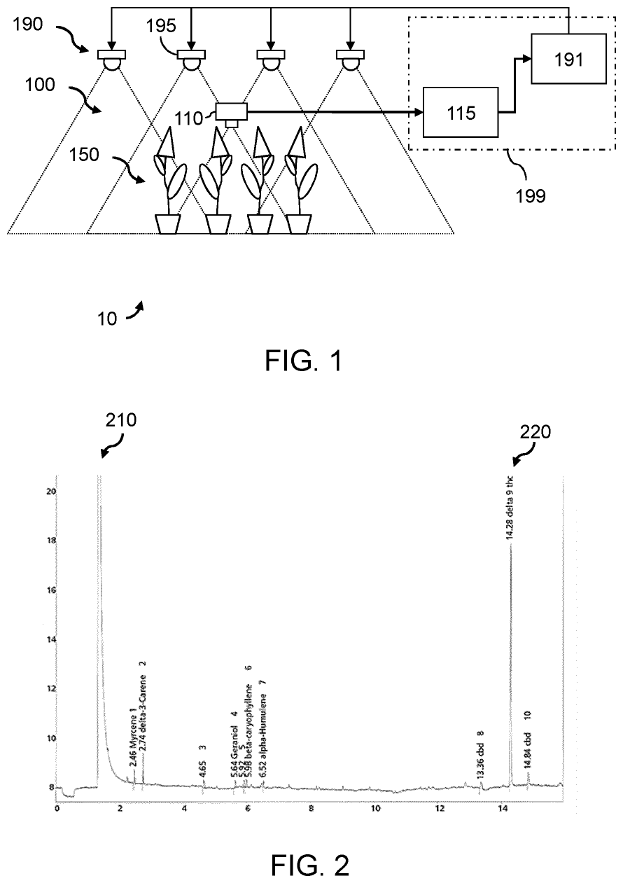

FIG. 1 illustrates a system 10 according to an embodiment of the invention. The system 10 comprises a cannabis monitoring system 100 and an (optional) environment modification system 190. The cannabis monitoring system 100 is itself an embodiment of the invention.

The cannabis monitoring system 100 is configured to monitor cannabis 150, e.g., cannabis plants. The cannabis monitoring system comprises a sensor arrangement 110 and a processing arrangement 115.

The sensor arrangement 110 is configured to monitor, over a period of time, an amount of a first set of one or more volatile organic compounds, VOCs, in air within the vicinity of the cannabis 150. This generates a first temporal sequence of monitored VOC values. In the first temporal sequence, each VOC value represents a measure of the first set of one or more VOCs at a different point in time.

In this way, the sensor arrangement acts as a VOC sensor that is able to detect volatiles emitted into the air by the plants, especially the flowers of the plants.

The sensor arrangement 110 may, for instance, be configured to detect or monitor an amount of all VOCs in the air within the vicinity of the cannabis 150. Such a sensor arrangement may comprise, for instance, the relatively low cost SGP30 sensor from company Sensirion, although other VOC detection systems will be apparent to the skilled person. Thus, the first set of one or more VOCs may comprise all detectable VOCs.

In a more advanced set up, the sensor arrangement 110 may be configured to discriminate between different types of VOCs. For instance, the sensor arrangement 110 may be configured to identify an amount of particular VOCs (such as those produced by a flowering cannabis plant, such as monoterpenes (e.g., Myrcene) and sesquiterpenes (e.g., Caryophyllene). Thus, the first set of VOCs may comprise a particular subset (i.e., not all) of all detectable VOCs.

One suitable example of a suitable discriminative VOC detector for the sensor arrangement 110 of the more advanced set up is a gas-chromatography (GC) sensor. A GC sensor is able to generate (for each time step) a signature or spectrum containing peaks, were the peak position, e.g., in terms of molecular mass or retention time, is indicative of the type of volatile compound and the peak height (or peak surface area) is proportional to the concentration of this volatile compound being present in the air.

FIG. 2 illustrates an example signature generated by a GC sensor. Each peak 210, 220 in the signature corresponds to a different VOC. It is therefore possible to identify an amount of a particular or specific VOC in the air within the vicinity of the cannabis.

The processing arrangement 115 is configured to determine, based on (i.e., by processing) the first temporal sequence of VOC values, a harvesting time for the monitored cannabis.

A harvesting time is a time at which the cannabis is predicted to meet some predetermined criteria for harvest, e.g., contain a certain or no lower than a predetermined minimum amount or proportion of a desired substance and/or no higher than a predetermined maximum amount or proportion of a substance (e.g., for clinical needs or medication). In particular, the harvesting time may be a predicted or estimated time at which the cannabis contains the maximum possible amount of that desired substance for harvesting, i.e., the harvesting time may be the optimum harvesting time.

This relies upon the recognition that, as the cannabis matures and its flowers bloom, a number of VOCs released by the cannabis plant/flowers increases. It is therefore possible to identify a harvesting time—i.e., a time at which the cannabis is harvest-ready or an optimum time for harvest—by tracking the amount of VOCs in the air within the vicinity of the cannabis.

One advantage of using a first temporal sequence of VOC values to determine the harvesting time is that, when changing the growth conditions (such as a photoperiod), the first temporal sequence of VOC values can still be used to determine or predict the harvesting time, without the need for recalibration. This is because the VOC values are proportional to the volatile compound composition of the flowers.

In particular, it is recognized that a trend or temporal pattern/change of (e.g., particular) VOCs values can be used to predict, estimate or determine the harvesting time. A temporal pattern is a pattern over time. In other words, a particular trend or temporal pattern represents a progression in the volatile compound composition of the cannabis towards harvesting time, and can therefore be used to predict or determine the harvesting time.

Thus, the processing arrangement may be able to determine or predict the harvesting time using a trend or temporal pattern in the first temporal sequence of VOC values.

This may be performed, for instance, by extrapolating the first temporal sequence to identify a time at which a measured VOC value is expected to reach a predetermined value (indicating the cannabis plant is harvest ready). This predetermined value may be determined from calibration experiments.

An alternative approach is to process a temporal pattern of VOC values using a machine-learning method to identify a predicted harvesting time. A machine-learning algorithm is any self-training algorithm that processes input data in order to produce or predict output data. Suitable machine-learning algorithms for being employed in the present invention will be apparent to the skilled person. Examples of suitable machine-learning algorithms include decision tree algorithms and artificial neural networks. Other machine-learning algorithms such as logistic regression, support vector machines or Naïve Bayesian models are suitable alternatives.

Methods of training a machine-learning algorithm are well known. Typically, such methods comprise obtaining a training dataset, comprising training input data entries and corresponding training output data entries. An initialized machine-learning algorithm is applied to each input data entry to generate predicted output data entries. An error between the predicted output data entries and corresponding training output data entries is used to modify the machine-learning algorithm. This process can be repeated until the error converges, and the predicted output data entries are sufficiently similar (e.g., within ±1%) to the training output data entries. This is commonly known as a supervised learning technique.

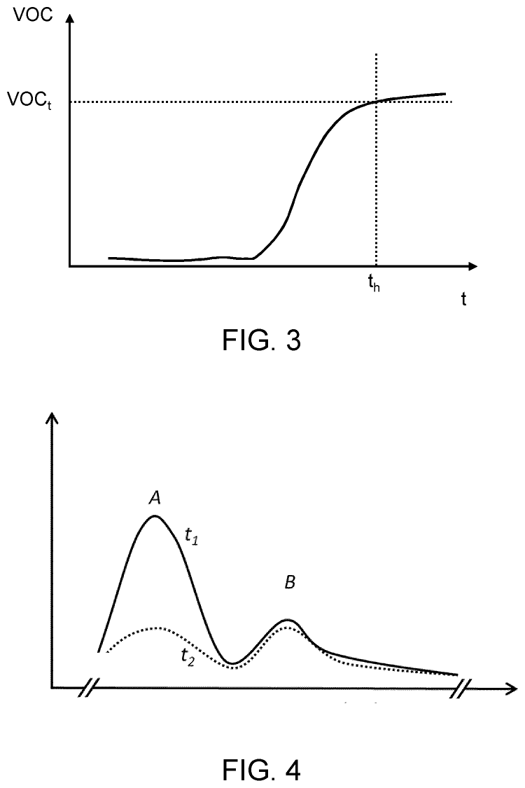

FIG. 3 is a graph illustrating a relationship between total amount of VOCs (VOC) released by cannabis plants/flowers and time (t). At a harvesting time tₕ, the total amount of VOCs exceeds some predetermined threshold VOCₜ. Thus, it is possible to predict or define a harvesting time based on a trend in a measured amount of VOCs.

For instance, the time series data of the first temporal sequence of VOC values may be extrapolated to predict or estimate a harvesting time, i.e., a point at which the measured VOCs will or is likely to exceed some predetermined threshold.

FIG. 3 demonstrates how the temporal data from a sensor arrangement comprising a simple, non-specific VOC sensor (that detects all VOC compounds) can be used to determine a harvesting time.

However, further embodiments recognize that more accurate identification of the harvesting time can be achieved using measurements of specific VOCs, i.e. VOCs specific to cannabis plants/flowers. For instance, rather than the first temporal sequence containing measures of all VOC compounds, the first temporal sequence may instead contain a measure of only a specific sub-set of (i.e., not all) VOC compounds.

This specific sub-set of VOC compounds in the first set of VOC compounds may include one or more terpenes and/or cannabinoids, e.g., may only include such one or more terpenes and/or cannabinoids. Suitable examples of terpenes include monoterpenes and/or sesquiterpenes released by cannabis flowers, e.g., Cyrcene and/or Caryophyllene.

Yet further embodiments recognize that a change in proportion of or a change is a certain relationship between particular VOC compounds is indicative of the cannabis plant/flower becoming harvest ready. For instance, it is recognized that a ratio of monoterpenes to sesquiterpenes changes during the course of flower maturation, i.e., from early flowering (majority of sesquiterpenes) to the end of flowering (majority of monoterpenes).

Thus, in some examples, the sensor arrangement 110 may be configured to detect over the period of time, an amount of a second (different) set of one or more volatile organic compounds, VOCs, in air within the vicinity of the cannabis. This generates a second temporal sequence of monitored VOC values, each VOC value of the second temporal sequence temporally corresponding to a respective VOC value of the first temporal sequence and representing a measure of the second set of one or more VOCs.

For instance, the sensor arrangement 110 may comprise a GC sensor that is able to discriminate between different VOCs, as has been previously described.

The processing arrangement 115 may be correspondingly configured to predict or determine a harvesting time based on the first temporal sequence and the second temporal sequence.

In some examples, trends in both the first and second temporal sequences are used to determine the harvesting time. For example, a harvesting time may be predicted to occur when a predicted value for a first set of one or more VOCs is to reach at least a first value (e.g., based on historic trends) and when a predicted value for a second set of one or more VOCs is to reach at least a second value (e.g., based on historic trends).

In an advanced embodiment, a third temporal sequence is produced by processing the first and second temporal sequences. Each value in the third temporal sequence may temporally correspond to a value in the first and second temporal sequences and may be derived from the corresponding values.

In a preferred example, each value in the third temporal sequence corresponds to a ratio between the corresponding values in the first and second temporal sequence. In this way, if the first temporal sequence is a temporal sequence of monitored values for VOC “A” and the second temporal sequences is a temporal sequence of monitored values for VOC “B”, then the third temporal sequence may be a temporal sequence of A/B or B/A. Over time, when approaching the time of harvest, the ratio A/B for the compounds “A” and “B” will change. The determined or predicted harvesting time then is that time at which this ratio is expected to equal a certain value, e.g., as obtained from calibration experiments. It is therefore possible to predict or determine a harvesting time based on trends of the third sequence of values.

VOC “A” may be a monoterpene such as Myrcene. VOC “B” may be a sesquiterpene such as Caryophyllene. Other suitable compounds (for monoterpenes and/or sesquiterpenes) will be apparent to the skilled person. This approach recognizes that the ratio in VOC values between monoterpenes and sesquiterpenes released by cannabis plants/flowers changes between the early flowering (majority of sesquiterpenes) and the end of flowering (majority of monoterpenes), and is therefore a good example of a value responsive to maturation and/or growth of the cannabis, and therefore a good example of how harvesting time can be determined/predicted.

It has been identified that use of a ratio in this way provides a particularly robust and effective mechanism for identifying time of harvest, as it is less sensitive to the actual amount or magnitude of VOCs in the air surrounding the cannabis (which may change subject to, for instance, size of the cannabis plant and/or environmental conditions).

However, in other approaches, each value in the third temporal sequence may be generated in another way, e.g., by performing a weighted sum or a weighted average of the corresponding first and second values.

Although in this example only two values (from the first and second temporal sequences) are used to generate a value for the third temporal sequence, the skilled person would appreciate that more advanced examples may make use of more than two values to produce each value in the third temporal sequence.

Thus, more generally, there may be a plurality of temporal sequences, each of which has temporally corresponding values. For each temporal sequence, each value may represent a measure of a respective set of one or more VOCs. A combined temporal sequence may be generated from the plurality of temporal sequences and used to predict or determine a harvesting time (e.g., based on trends in the combined temporal sequence). For instance, values of the combined temporal sequence may be extrapolated to identify a harvesting time, e.g., a time at which the extrapolated value reaches a predetermined measure or value indicating harvest-readiness—which may be determined by calibration experiments or the like.

The combination of the plurality of temporal sequences may comprise any number of summing, averaging, subtracting, dividing or multiplying steps of respective corresponding VOC values.

In yet other embodiments, the first temporal sequence may contain (for each entry in the temporal sequence) a signature or spectrum produced by a multi-VOC detecting sensor, such as a GC sensor. The signature may indicate for each of a plurality of different VOCs, a measure of that VOC. The signature may be on a continuous scale (such as a molecular mass scale), for example.

An example of a suitable signature is illustrated in FIG. 4, which represents the output of a GC sensor at two points in time: t₁ (solid line) and t₂ (dotted line). These may represent different time points during the maturation of the cannabis flower. FIG. 4 also indicates two peaks (representing two particular VOC compounds “A” and “B”).

FIG. 4 clearly demonstrates how a signature can change over time and how a signature can be used to predict a harvesting time. In particular, at a harvesting time, such signature will match some predetermined pattern or “fingerprint”, which will be near-unique to the harvesting time and correspond to a harvest-ready cannabis plant/flower. In this way, the approaching of such VOC fingerprint can effectively act as an indicator for the approaching harvesting time.

Thus, it is possible to process a sequence of signatures in order to predict or determine a harvesting time. This may be performed, for instance, using machine-learning algorithms to process the temporal sequence of signatures in order to predict or determine the harvesting time.

FIG. 4 further illustrates how a ratio between compound “B”, e.g., a monoterpene such as Myrcene, and compound “A”, e.g., a sesquiterpene such as Caryophyllene, in VOCs released by cannabis plants/flowers changes over time. In particular, this ratio changes between the early flowering (majority of sesquiterpenes) and the end of flowering (majority of monoterpenes). It has been previously explained how this ratio could be exploited to identify a harvesting time according to some embodiments.

In any above described embodiment, improved reliability and accuracy of the determination of the harvesting time can be achieved by employing one or more approaches described hereafter. These approaches restrict or define when a VOC value for a temporal sequence is to be obtained or how to improve the accuracy or representativeness of a VOC value. The following approaches may be applied to any herein described temporal sequence (e.g., the first temporal sequence, the second temporal sequence or the plurality of temporal sequences).

An amount of VOCs released by a cannabis plant/flower can be affected by air temperature. Generally, the higher the temperature, the more VOCs are released. Thus, a temporal sequence of VOC values may be configured to only contain VOC values obtained at a predetermined temperature or within a predetermined temperature range or at a predicted temperature or within a predicted temperature range.

In some examples, each temporal sequence may only contain VOC values obtained within a predetermined temperature range (e.g., as detected by a temperature sensor).

In other examples, each temporal sequence may contain VOC values obtained during daylight hours only or nighttime hours only (e.g., as detected by a light sensor or a clock co-operating with known sunrise/sunset times). This approach is advantageous because a temperature difference between different times within the day or within the night (as well as between different days and different nights) is less than a temperature difference between the day and the night.

It is also recognized that flowers at different heights of a cannabis plant may have different stages of development. Thus, in some examples, the sensor arrangement may be configured to monitor VOC values at different heights and each temporal sequence may contain averaged VOC values, where an averaged VOC value is an average of VOC values at different heights. This approach provides VOC values that provide a more appropriate and plant representative indicator of harvest-readiness.

The concentration of volatiles in the air could be enhanced by active manipulation of the plant or flowers, such as by sonication (the application of sound, e.g., close to the resonance frequency of the trichomes on the flowers to disturb—to some extent—the flowers) or vibration. Thus, in some embodiments, each sequence of VOC values may only contain values obtained after a respective manipulation, such as a respective insonification and/or vibration, of the cannabis.

In some examples, the cannabis monitoring system 100 (illustrated in FIG. 1) may comprise an insonification and/or vibration system, configured to manipulate (e.g., through (in)sonification and/or vibration) the cannabis 150 before a VOC value is measured/monitored. This approach enhances the concentration of VOCs in the air surrounding the cannabis, thereby improving a signal to noise ration of measured VOCs.

With continued reference to FIG. 1, it has been previously described how the system 10 may comprise an environment modification system 190.

It is recognized that cannabis growers may prefer to have the harvest at a predetermined date or time, e.g., to synchronize the growth cycles of different cannabis varieties and/or for logistical reasons and/or for market demand reasons. It is herein proposed to steer the growth of the cannabis by changing/modifying the environment, to thereby control the harvesting time to be closer or equal to a desired harvesting time.

In this way, the environment modification system 190 may effectively operate within a feedback system. More specifically, the environment surrounding the cannabis plants 150 may be controlled by an environment controller 191 responsive to the determined/predicted/estimated harvesting time (e.g., determined by the processing system 115) and the desired harvesting time (e.g., input to the environment controller 191 by a user or other processing system) to control a growth or maturation rate of the cannabis plant/flower to align the determined/predicted/estimated harvesting time to the desired harvesting time.

Various approaches for controlling an environment to thereby control a growth/maturation rate of the cannabis plant/flower are envisaged. In particular, one or more properties of a growth environment may be controlled.

The one or more properties of the growth environment comprise a temperature, a moisture level, a spectrum of light, a duration of light and/or a magnitude of light. Properties of light may be controlled, for instance, by controlling light emitting elements 195 (e.g., an LED based horticulture light arrangement) positioned to provide light to the cannabis plants. Other properties may be modified using any suitable environment modifying device, such as a temperature-controlling arrangement (e.g., air-conditioning or cooling systems and/or heating systems), a humidity controller (e.g., a humidifier and/or a dehumidifier) and so on.

For instance, if the cannabis plant/flowers are lagging in their development at any given time before harvest, the temperature may be slightly increased and/or the light level (in duration and/or magnitude of light) may be increased. Another example is to increase the magnitude or proportion of blue light in a light spectrum provided to the cannabis (e.g., where blue light is defined as electromagnetic radiation having a wavelength of between 400-500 nm, although other definitions will be apparent to the skilled person). These approaches would accelerate the development of the cannabis plant/flower. As another example, it may for instance be assumed that a cannabis flower will be ready for harvest after a fixed amount of degree-days (being the integral of the temperature (minus a temperature offset) over time).

Of course, if the cannabis plant/flower is developing faster than desired (e.g., the harvesting time is predicted to fall before the desired harvesting time), opposite measures may be taken.

The environment controller 191 and the processing arrangement 115 of the cannabis monitoring system may be integrated into a same processing system 199 (e.g. a computer or computer array).

It will be appreciated that the system 10 may comprise a plurality (not shown) of different cannabis monitoring systems for monitoring cannabis at different positions within a growth area. The environment modification system may similarly operate on a modular basis to control the growth of cannabis in different locations.

The skilled person will be readily capable of defining a method for carrying out any above-described approach.

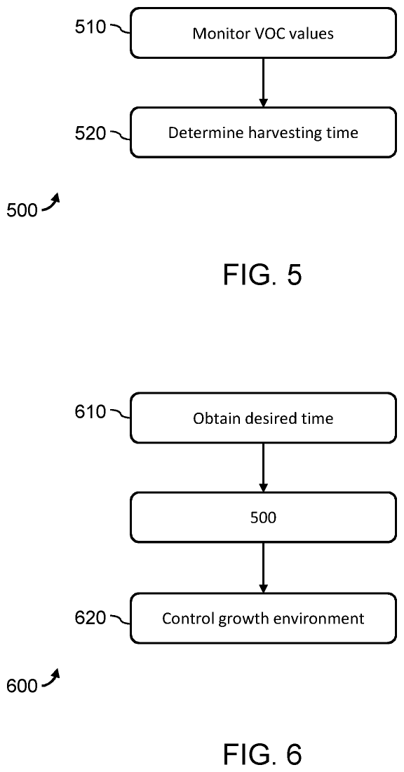

For the sake of completeness, FIG. 5 illustrates a method 500 according to an embodiment.

The method 500 comprises a step 510 of monitoring, using a sensor arrangement and over a period of time, an amount of a first set of one or more volatile organic compounds, VOC, in air within the vicinity of the cannabis. Step 510 thereby generates a first temporal sequence of monitored VOC values, each VOC value representing a measure of the first set of one or more VOCs at a different point in time.

The method 500 further comprises a step 520 of determining, based on the first temporal sequence of VOC values, a harvesting time for the monitored cannabis. More particularly, step 520 may comprise determining a harvesting time based on one or more temporal patterns within the first temporal sequence of VOC values.

In some examples, a method may comprise performing a plurality of instances of the method 500, each instance being performed at a different location and/or height of the crop of the one or more cannabis plants.

FIG. 6 illustrates a method 600 of controlling a growth environment for cannabis plants.

The method 600 comprises a step 610 of obtaining a desired time for harvesting the cannabis; performing a previously described method (e.g., method 500) to determine a harvesting time of the cannabis; and a step 620 of, responsive to a difference between the determined harvesting time and the desired time, modifying one or more properties of the growth environment for the cannabis plant. Step 620 may be performed using environment modifying devices, e.g., a lighting arrangement or any other environment modifying device previously described.

The one or more properties of the growth environment may comprise a temperature, a moisture level, a spectrum of light, a duration of light and/or a magnitude of light.

It will be understood that disclosed methods are preferably computer-implemented methods. For instance, the step of monitoring, using a sensor arrangement, may comprise communicating with or controlling the sensor arrangement via a sensor arrangement interface of a computer or processing system to performing a monitoring step.

As such, there is also proposed a computer program comprising program code for implementing any described method when said program is run on a processing system, such as a computer. Thus, different portions, lines or blocks of code of a computer program according to an embodiment may be executed by a processing system or computer to perform any herein described method.

A processing system can be implemented in numerous ways, with software and/or hardware, to perform the various functions required. A processor is one example of a processing system which employs one or more microprocessors that may be programmed using software (e.g., microcode) to perform the required functions. A processing system may however be implemented with or without employing a processor, and also may be implemented as a combination of dedicated hardware to perform some functions and a processor (e.g., one or more programmed microprocessors and associated circuitry) to perform other functions.

Examples of processing system components that may be employed in various embodiments of the present disclosure include, but are not limited to, conventional microprocessors, application specific integrated circuits (ASICs), and field-programmable gate arrays (FPGAs).

In various implementations, a processor or processing system may be associated with one or more storage media such as volatile and non-volatile computer memory such as RAM, PROM, EPROM, and EEPROM. The storage media may be encoded with one or more programs that, when executed on one or more processors and/or processing systems, perform the required functions. Various storage media may be fixed within a processor or processing system or may be transportable, such that the one or more programs stored thereon can be loaded into a processor or processing system.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. If a computer program is discussed above, it may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted the term “adapted to” is intended to be equivalent to the term “configured to”. Any reference signs in the claims should not be construed as limiting the scope.

1. A method for monitoring cannabis, the method comprising:
monitoring, using a sensor arrangement and over a period of time, an amount of a first set of one or more volatile organic compounds, VOCs, in air within the vicinity of the cannabis to thereby generate a first temporal sequence of monitored VOC values, each VOC value representing a measure of the first set of one or more VOCs at a different point in time; and
predicting, based on one or more temporal patterns within the first temporal sequence of VOC values, a harvesting time for the monitored cannabis.

2. The method of claim 1, wherein the step of predicting a harvesting time comprises:
extrapolating the first temporal sequence of VOC values to identify a time at which a measured VOC value is expected to reach a predetermined value indicative of the cannabis plant being harvest ready, or
using a machine-learning method to identify a predicted harvesting time.

3. The method of claim 1, wherein the first set of one or more volatile organic compounds comprises one or more terpenes and/or cannabinoids.

4. The method of claim 3, wherein the first set of one or more volatile organic compounds comprises only the one or more terpenes and/or cannabinoids.

5. The method of any of claim 3, wherein the first set of one or more volatile organic compounds comprises one or more monoterpenes and/or sesquiterpenes.

6. The method of any of claim 1, wherein the method further comprises:
monitoring, using the sensor arrangement and over the period of time, an amount of a second set of one or more volatile organic compounds, VOCs, in air within the vicinity of the cannabis to thereby generate a second temporal sequence of monitored VOC values, each VOC value of the second temporal sequence temporally corresponding to a respective VOC value of the first temporal sequence and representing a measure of the second set of one or more VOCs; and
processing the first and second temporal sequences to produce a third temporal sequence, each value of the third temporal sequence being temporally associated with a pair of respective values from the first temporal sequence and the second temporal sequence and being derived from the respective values of the first temporal sequence and the second.

7. The method of claim 6, wherein each value of the third temporal sequence is equal to the ratio of the respective value of the first temporal sequence and the second temporal sequence.

8. The method of claim 1, wherein the first set of one or more volatile organic compounds comprises all volatile organic compounds.

9. The method of claim 1, wherein the first temporal sequence of monitored VOC values comprises VOC values obtained within a predetermined temperature range only.

10. The method of claim 1, wherein each VOC value in the first temporal sequence of monitored VOC values is obtained after a respective manipulation, such as a respective insonification and/or vibration, of the cannabis.

11. A method of controlling a growth environment for cannabis, the method comprising:
obtaining a desired time for harvesting the cannabis;
performing the method of claim 1 to predict a harvesting time the cannabis; and
responsive to a difference between the predicted harvesting time and the desired time, modifying one or more properties of the growth environment for the cannabis.

12. The method of claim 11, wherein the one or more properties of the growth environment comprises a temperature, a moisture level, a spectrum of light, a duration of light and/or a magnitude of light.

13. A method of monitoring a crop of one or more cannabis plants, the method comprising performing a plurality of instances of the method of claim 1, each instance being performed at a different location and/or height of the crop of the one or more cannabis plants.

14. A non-transitory computer readable medium comprising instructions which, when the instructions are executed on a computing device having a processing system communicatively coupled with a sensor arrangement, causes the processing system to perform the method according to any of claim 1.

15. A cannabis monitoring system comprising:
a sensor arrangement configured to monitor, over a period of time, an amount of a first set of one or more volatile organic compounds, VOCs, in air within the vicinity of the cannabis to thereby generate a first temporal sequence of monitored VOC values, each VOC value representing a measure of the first set of one or more VOCs at a different point in time; and
a processing arrangement, communicatively coupled to the sensor arrangement, and configured to predict, based on one or more temporal patterns within the first temporal sequence of VOC values, a harvesting time for the monitored cannabis.
